(19) United States
(12) Patent Application Publication
(10) Pub. No.: US 2017/0224086 A1
(43) Pub. Date: Aug. 10, 2017

(54) RETRACTABLE DISPENSER AND APPLICATION HEAD

(71) Applicant: CLICK CO PRODUCTS LLC, Herriman, UT (US)

(72) Inventor: Ryan Relf, Sandy, UT (US)

(73) Assignee: CLICK CO PRODUCTS LLC, Herriman, UT (US)

(21) Appl. No.: 15/493,063

(22) Filed: Apr. 20, 2017

Related U.S. Application Data
(60) Provisional application No. 62/409,865, filed on Oct. 18, 2016.

(51) Int. Cl. A45D 34/04; A61M 35/00

(52) U.S. Cl. CPC A45D 34/041; A61M 35/003

(57) ABSTRACT

The invention relates to the application of topical fluids to the skin of humans. For example, the topical fluid can be a viscous oil, medication, fragrance, cosmetic, or other topical fluid. The improvements in functionality, simplicity, and use allow for a more simple, reliable and versatile applicator for easier application of topical fluids. A retractable and refillable topical fluid applicator device allows for a topical fluid applicator to be repositionable from a first retracted position within a housing thereby preventing accidental contact to a second protracted position at which the applicator head is exposed for application of topical fluid. Improved applicator heads are disclosed which include a biasing member urging a rotatable applicator ball into a sealed position in connection with a sealing ring disposed around a periphery of the applicator head. Kits containing the retractable applicator device and a plurality of topical fluid cartridges are also disclosed.

### CROSS-REFERENCE TO RELATED APPLICATIONS

This application claims the benefit of and priority to U.S. provisional patent application 62/409,865 filed Oct. 18, 2016 the contents of which are hereby incorporated by reference herein.

### BACKGROUND

Topical fluids including liquids, gels, viscous oils, ointments, and medications are generally applied directly to body surfaces such as the human skin or membranes. Applicators and application methods of such topical fluids can result in various complications. For example the applicator may leak, drip, provide uneven application, create undue waste, or cause inconvenience.

To this end, various prior art devices and applicator heads have been designed such as that disclosed in U.S. Pat. No. 7,311,462. Such designs have endeavored to provide a “roll on” type applicator head that enables a good level of tightness to be achieved between the ball and the reservoir container holding the material to be applied. To achieve this goal, the '462 patent describes a container having a portion designed to contain the product to be applied, and designed to be placed in fluid communication with a seating obtained by molding with the portion containing the product. The seating contains a spherical ball mounted freely in rotation inside the seating. And, an applicator portion of the ball emerges outwardly from the container through an opening formed by an edge of the seating. To prevent the leakage, a cap is designed to cover the applicator portion of the ball. When the cap is mounted on the container. The cap presses the ball inward in the leak tight manner against at least two annular sealing zones formed by the inner surface of the seating. An annular zone of the inner surface located between the two sealing zones is not in contact with the ball when the latter is bearing in a leak tight manner against the two annular sealing zones. However, as recognized by the inventors of this patent application this leak-tight arrangement does not remain without the cap in place nor if the cap does not securely engage the ball against the two annular sealing zones during assembly, during shipment, nor even during use. These shortcomings are particularly prevalent if the cap, ball, and annular sealing zones are inaccurately manufactured, assembled, or the cap is insecurely placed by the end user.

Another shortcoming of conventional applicators is that they are generally single use applicators. And, some conventional applicators may simply be made to be disposable. For example, the ball type application head and cap may be permanently affixed to a reservoir of liquid for topical application but not easily replaceable or refillable. And, where application of a different topical fluid is required, or desired, the replacement of such in an accessible applicator may not be available.

According to the various embodiments and improvements discussed hereinafter new and innovative full-face mask and snorkel designs and processes associated thereto are disclosed and set forth in the claims. The subject matter claimed herein is not limited to embodiments that solve any disadvantages or that operate only in environments such as those described above. Rather, this background is only provided to illustrate examples of the technology area where some embodiments described herein may be practiced and find certain advantages.

### SUMMARY

This Summary is provided to introduce a selection of concepts in a simplified form that are further described below in the Detailed Description. This Summary is not intended to identify key features or essential characteristics of the claimed subject matter, nor is it intended to be used as an aid in determining the scope of the claimed subject matter.

The invention relates to the application of topical fluids to the skin of humans. For example, the topical fluid can be a viscous oil, medication, fragrance, cosmetic, or other topical fluid. The improvements in functionality, simplicity, and use allow for a more simple, reliable and versatile applicator for easier application of topical fluids.

A retractable topical fluid applicator device is disclosed. The retractable topical fluid device includes a housing and a receptacle for holding a topical fluid applicator cartridge. The receptacle is configured for reciprocation within the housing between protracted and retracted positions. The retractable topical fluid applicator device includes a spring biasing the receptacle in the retracted position. The retractable topical fluid applicator device includes a push member for moving the reciprocating receptacle into the protracted position. The retractable topical fluid applicator device includes a mechanism for holding the receptacle in the protracted position and for releasing the receptacle from the protracted position to return to the retracted position. The retractable topical fluid applicator device includes a seal disposed at an end of the housing. The seal can comprise one or more slits in a silicone or other resilient material through which the applicator is allowed to extend in the protracted position. The slit can extend across the end of the applicator and two slits can be formed in the shape of an X according to some embodiments.

The retractable topical fluid applicator device can further include the topical fluid applicator cartridge held by the receptacle, wherein the topical fluid applicator extends through the seal in the protracted position and is covered by the seal in the retracted position. The topical fluid applicator cartridge can be held by a slip engagement with the receptacle for replacement with a different topical fluid applicator cartridge. The slip engagement may include a slight interference fit. Other options include a rotationally keyed fitting or other means for selectively securing the cartridge within the receptacle. The topical fluid applicator cartridge can include a topical fluid applicator head, a reservoir, and a viscous oil contained within the reservoir. The retractable topical fluid applicator device can be defined by a height of the topical fluid applicator device of at least 60 millimeters and a width of the topical fluid applicator device is at least 15 millimeters.

The seal can include the resilient slit having sealing lips extending substantially across a diameter of the width of the seal thereby creating a displaceable opening for the topical fluid applicator to extend through in the protracted position and a sealed interface between the lips when the topical fluid applicator is in the retracted position.

The push member can be affixed via channels complementing protrusions along an interior of the housing rendering the push device non-rotatable. The push member includes downwardly projecting teeth and a mechanism for holding the receptacle in the protracted position and for releasing the receptacle from the protracted position including a rotatable ratchet interlocking member. The rotatable ratchet member has upwardly projecting teeth which selectively engage the downwardly projecting teeth of the push member. The ratchet member is disposed between the spring and the push member whereby the spring forces the rotatable ratchet member against the teeth of the push member, at least one of the engaging surface of the teeth of the push member and the rotatable ratchet member being inclined so that the force of engagement between the teeth of the push member and the engaging surface of the rotatable ratchet member caused by the spring rotatably biases the rotatable member ratchet member for selective transition between the protracted and retracted positions by successive manual manipulations of the push member.

A topical fluid applicator head is disclosed. The topical fluid applicator head includes a barrel having an interior surface and an outer surface. The topical fluid applicator head further includes a support appendage extending around a periphery of the cylindrical interior surface. The topical fluid applicator head further includes an applicator ball disposed within the barrel and supported by the annular support appendage of the barrel. The topical fluid applicator head further includes a sealing ring disposed around an upper periphery of the ball and a can. The can includes an inner surface corresponding to the outer surface of the barrel and an annular lip extending over a top surface of the sealing ring thereby holding the sealing ring against the applicator ball and exposing a portion of the applicator ball for application of the topical fluid. The topical fluid applicator head further includes a biasing member, the biasing member urging the applicator ball against the sealing ring.

The topical fluid applicator head can include a holder disposed between the biasing member and the applicator ball, the biasing member pressing the holder against a lower surface of the applicator ball. The sealing ring creates an annular seal around an upper periphery of the applicator ball. And, the biasing member can be a wave washer. The cap extends over the applicator ball without contacting the applicator ball.

The topical fluid applicator head can further include an insertion sleeve extending below the annular support for interference mounting the barrel to a reservoir. The topical fluid applicator head can further include a reservoir containing topical fluid, the reservoir being interference fit to the insertion sleeve, the reservoir providing the topical fluid to the applicator ball for rotational application of the fluid to the skin of a human.

A kit is disclosed comprising a case, a retractable topical fluid applicator device, and a plurality of cartridges containing topical fluid. The plurality of the topical fluid applicator cartridges for interchangeable placement in the receptacle of the retractable topical fluid applicator device.

Additional features and advantages of the invention will be set forth in the description which follows, and in part will be obvious from the description, or may be learned by the practice of the invention. The features and advantages of the invention may be realized and obtained by means of the instruments and combinations particularly pointed out in the appended claims. These and other features of the present invention will become more fully apparent from the following description and appended claims, or may be learned by the practice of the invention as set forth hereinafter.

### BRIEF DESCRIPTION OF THE DRAWINGS

To further clarify the above and other advantages and features of the present invention, a more particular description of the invention will be rendered by reference to specific embodiments thereof which are illustrated in the appended drawings. It is appreciated that these drawings depict only typical embodiments of the invention and are therefore not to be considered limiting of its scope. The invention will be described and explained with additional specificity and detail through the use of the accompanying drawings in which:

FIG. 1 illustrates a topical fluid applicator as well as methods for manufacture and assembly thereof;

FIGS. 2A and 2B illustrate a topical fluid applicator head;

FIGS. 3A, 3B, 3C, and 3D show a rechargeable topical fluid applicator from a retracted position shown in FIG. 3A, to a protracted position shown in FIG. 3B, and back into the retracted position shown in FIGS. 3C and 3D;

FIG. 4A shows the exterior of the rechargeable topical fluid applicator;

FIGS. 4B and 4C show the internal components of the topical fluid applicator;

FIG. 5 shows an exploded view of the rechargeable topical fluid applicator along with a replaceable cartridge having a reservoir and application head; and

FIG. 6 illustrates a kit having a retractable topical fluid applicator device, a plurality of topical fluid applicator cartridges for interchangeable placement in the retractable topical fluid applicator device and a case.

### DETAILED DESCRIPTION ILLUSTRATING THE INVENTION

The invention relates to the application of topical fluids to the skin of humans. For example, the topical fluid can be a viscous oil, medication, fragrance, cosmetic, or other topical fluid. The improvements in functionality, simplicity, and use allow for a more simple, reliable and versatile applicator for easier application of topical fluids.

Various embodiments relate to improvements to the head of the topical applicator. Previously, the head of the topical applicator was often prone to leakage and dripping. Various embodiments of the prior art meant to solve this problem with a cap that presses on an applicator ball so as to prevent leakage while in transport but failed to prevent leakage once the cap is removed and the pressure against the applicator ball is no longer applied. Various embodiments disclosed herein create a novel biasing arrangement for holding the application ball in a constant forward and sealed position within the application head.

Thus, previous designs also required a removable cap so as to secure the otherwise leaking application head in a sealed transportable position. Due to the lack of the removable cap design disclosed herein, a removable cap is no longer a requirement. Rather, due to the seal created by the biased member, topical fluid applicator may be repositionable from a first retracted position within a housing thereby preventing accidental contact to a second protracted position at which the applicator head is exposed for application of topical fluid without the requirement of the removal and replacement of a cap.

Various sealable covers are disclosed for selectively exposing the applicator head. These covers can be made of a resilient sealing material that allows for the application head to extend through the seal in the second protracted position and also forms a substantially sealed covering when the topical fluid applicator is retracted within the housing.

As disclosed herein, the overall application unit can comprise a receptacle for holding a replaceable topical fluid applicator and reservoir. The topical fluid and applicator held by the receptacle can be easily replaced by a new topical fluid held reservoir once the original topical fluid is depleted. Similarly, the topical fluid and applicator held by the receptacle can be easily replaced by a different topical fluid held reservoir if application of the different topical fluid or different applicator head is desired for any reason, various examples of such reasons are disclosed herein or would be made known in view of this disclosure to one of ordinary skill in the topical fluid arts such as the application of viscous (e.g. “essential”) oils, medications, fragrances, cosmetics, or by those familiar with other topical fluids.

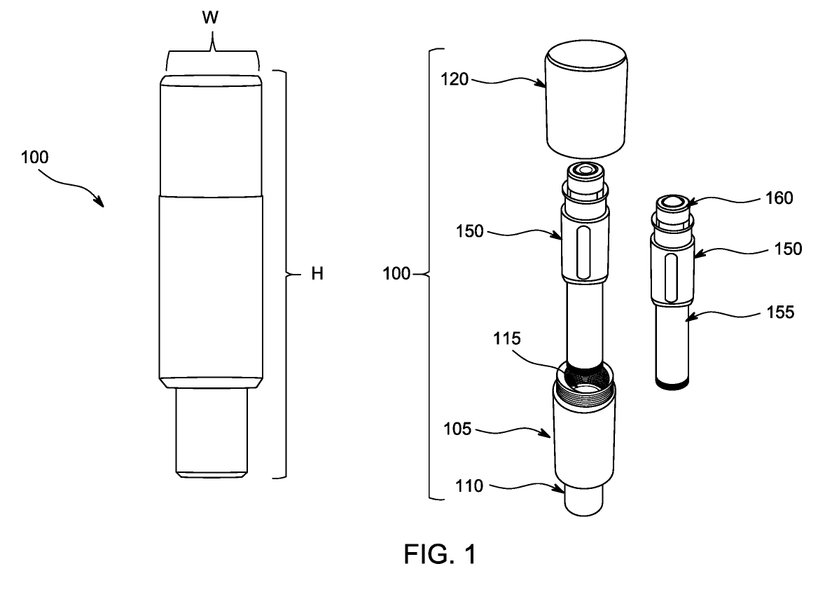

FIG. 1 illustrates a topical fluid applicator device 100 according to an embodiment of the invention. Additional methods for manufacture and assembly thereof are shown and described herein. The topical fluid applicator device 100 includes a rechargeable topical fluid application device including a housing 105. Within the housing 105 are various components discussed in detail below including a receptacle 115 that extends into a push member 110 extending from a lower end of the housing 105. The receptacle 115 extending within the housing 105 and push member 110 provide for placement of replaceable topical fluid cartridges 150. The topical fluid cartridges 150 can include a reservoir 155 such as a measured vial of topical fluid affixed to an applicator head 160. The topical fluid applicator device 100 can also include a cap or lid 120 that is detachably affixed to the housing 105 upon placement, or replacement of the topical fluid cartridge 150 therein. In this example the vial 155 of the topical fluid cartridge 150 is sized so as to slidably secure the topical fluid cartridge 150 within the housing 105 in a secure interference fit arrangement. Placement of the topical fluid cartridge 150 within the housing 105 substantially prevents mechanical movement of the topical fluid cartridges 150 within the housing 105 but for manual articulation using the push member 110 or upon manual replacement therein by a subsequent topical fluid cartridge 150. Thus, in this embodiment, the cap 100 and housing 105 include opposable threads for securing the cap 100 to the housing 105 and removal therefrom for replacement of the topical fluid cartridge 150 only as opposed to removal therefrom during use. Rather, the removable cap includes access means for allowing the applicator end 160 of the topical fluid cartridge 150 to extend there through during manual protraction and retraction thereof using the push element 110.

As shown in FIG. 1, the size of the topical fluid applicator device 100 can be defined by the type of topical fluid contained therein. For example, the topical fluid applicator device 100 can be defined by a height H and a width W. The height and width can relate in form to the size of the topical fluid cartridge 150 as the topical fluid cartridge is designed to extend into the push member and is held by the receptacle 115 as discussed in further detail below. For example, in the viscous oil applicator device 100 the volume of viscous oil held by the fluid cartridges 150 within the topical fluid applicator device 100 can be between 2 and 7 milliliters. More specifically, there can be a 2.7 milliliter embodiment measuring approximately 70 millimeters in height H and 17 millimeters in width W. A 3 milliliter embodiment can measure approximately 76 millimeters in height H and 20 millimeters in width W. And, a 5 milliliter embodiment may measure approximately 86 millimeters in height H and 22 millimeters in width W. The increase in height H and width W of the topical fluid applicators 100 according to the embodiments disclosed herein may only increase the overall height and width of the applicator device 100 over a conventional non-rechargeable disposable applicator by 10%-20% according to some embodiments.

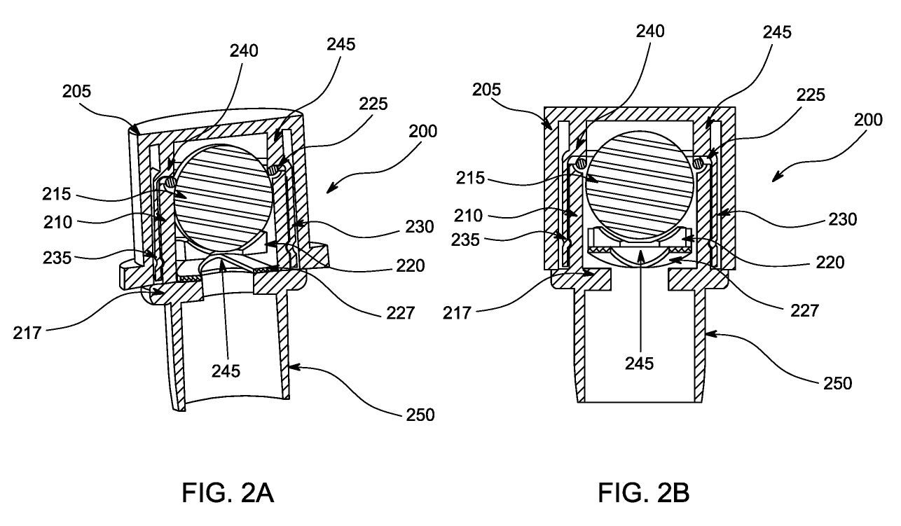

FIGS. 2A and 2B are cross sectional views of a topical fluid applicator head 200 according to an embodiment of the invention. As previously discussed the topical fluid applicator head 200 does not require a lid 205, however a temporary lid is shown for assembly thereto prior to use and during transport so as to protect the applicator and so as to prevent accidental contact. The lid may also be used to press downward against the top of the topical fluid applicator head during assembly with a topical fluid reservoir without pressing directly against the applicator ball 215.

The topical fluid applicator head 200 includes a barrel 210. The barrel 210 includes an interior surface and an outer surface. The barrel 210 includes a support appendage 217 extending around an interior and exterior periphery of the cylindrical interior and exterior surface of the barrel 210.

The topical fluid applicator head 200 further includes an applicator ball 215 disposed within the barrel 210 and supported by the annular support appendage 217 of the barrel 210 via a biasing member 227 and holder 220. The biasing member 225 urges the applicator ball holder 220 and applicator ball 215 upwards away from the support appendage 217 and against a sealing ring 225. The sealing ring 225 is disposed around an upper periphery of the applicator ball 215 creating a seal there between due to the consistent pressure between the sealing ring 225 and the application ball 215 applied by the biasing member 100.

In this example, the biasing member 100 is in the form of a wave washer. The wave washer 100 also called spring washers, feature a lobed design suitable for low strength fastening, irregular surfaces, and variations in tolerance. A typical washer is a thin plate (typically disk-shaped) with a hole (typically in the middle) that is normally used to distribute the load of a threaded fastener, such as a screw or nut. The wave washer 227 (belleville washer, spring washer) is used herein to produce the biasing force from the support appendage 217 to the applicator ball 215 via the ball holder 220 and from the applicator ball 215 against the sealing ring 225. The sealing ring 225 creating an annular seal around an upper periphery of the applicator ball 215. Another type of biasing member can be a curved disc, however the wave washer exhibits a more even distribution of biasing due to at least three depressed areas as opposed to only two depressed areas of the curved washer thereby creating a sinusoidal or wave-like perspective as viewed from its side.

The wave washer 227 can have an outer diameter (OD) conforming to the inter surface of the barrel 210 and an inner diameter (ID) approximately half the outer diameter or determined in size so as to allow sufficient flow of topical fluid there through. The sufficient flow of topical fluid through the wave washer 227 and through the application ball holder 220 to the application ball can be referred to as a topical fluid access channel 245 and provides the application ball with sufficient fluid for topical application during rotation of the applicator ball 215.

The topical fluid applicator head 200 further includes a can 230. The can 230 substantially covers the outer surface of the upper portion of the barrel 210 within which the application ball 215 is disposed. The can 230 includes an inner surface corresponding to the outer surface of the upper portion of the barrel 210. The upper and lower portions of the barrel 210 being defined by portions of the barrel 210 above and below the annular appendage 217. The can 230 includes a locating feature 235. The locating feature can include a crimp, key, or indentation in this example so as to hold the can 230 located in-place relative to the barrel 210 when assembled. The locating feature 235 can be made prior to assembly or may be made upon assembly of the can 230 with the barrel 210.

The can 230 includes an annular lip 240 extending over a top surface of the sealing ring 225 thereby holding the sealing ring 225 against the applicator ball 215. The annular lip 240 of the can 230 can define an upper opening of the can 230 through which an upper application portion of the applicator ball 215 extends for application of the topical fluid received through the fluidic access channel 245 created by the inner apertures of the annular appendage 217, holder 220 and biasing means 227. Thus the annular lip 240 partially, but not entirely, covers the applicator ball 215 so as to compress the sealing ring 225 against an upper annular surface of the applicator ball 215.

As previously discussed, the temporary cap 205 extends over the applicator ball 215 without contacting the applicator ball 215. The cap 205 can be in place and include guards 245 that contact the annular lip 240 as opposed to the applicator ball 215. The temporary cap 205 can protect the applicator ball 215 and cap 230 until assembled with a fluid reservoir (not shown) at which point the temporary cap may be used to press the application head 200 into a reservoir.

For example, the barrel 210 of the applicator head 200 can include an insertion sleeve 250 extending below the annular support 217 for interference mounting the application head 200 to a reservoir (not shown). Thus, when the insertion sleeve 250 is inserted into an upper end of a reservoir containing topical fluid. The reservoir's interference fit to the insertion sleeve 250 provides the topical fluid to the applicator ball 215 through the fluidic access channel 245 created by the inner apertures of the annular appendage 217, holder 220, and biasing means 227.

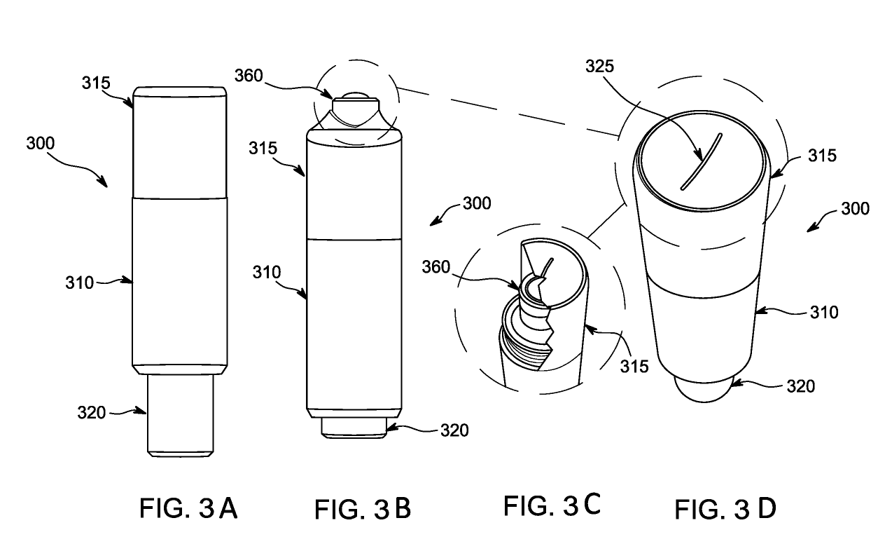

FIGS. 3A, 3B, 3C, and 3D illustrate a rechargeable topical fluid applicator 300. The topical fluid applicator 300 has a replaceable topical fluid cartridge disposed therein. The head 360 of the topical fluid applicator 300 is shown in a protracted position in FIG. 3B extending through an access slit in the top of a resilient cap 315 attached to the housing 310 of the topical fluid applicator 300. Two slits in the form of an X can also be used although there can be advantages to a single slit and the double slit embodiments. The head 360 of the topical fluid applicator 300 is shown in a retracted position in FIGS. 3A, 3C, and 3D where the application head 360 is contained within the housing 310 and cap 315 of the topical fluid applicator 300.

The rechargeable topical fluid applicator 300 includes a push member 320. The push member is used to manually move the head 360 of the cartridge held within the topical fluid applicator 300 from the retracted position shown in FIG. 3A to the protracted position shown in FIG. 3B and back into the retracted position shown in FIGS. 3C and 3D.

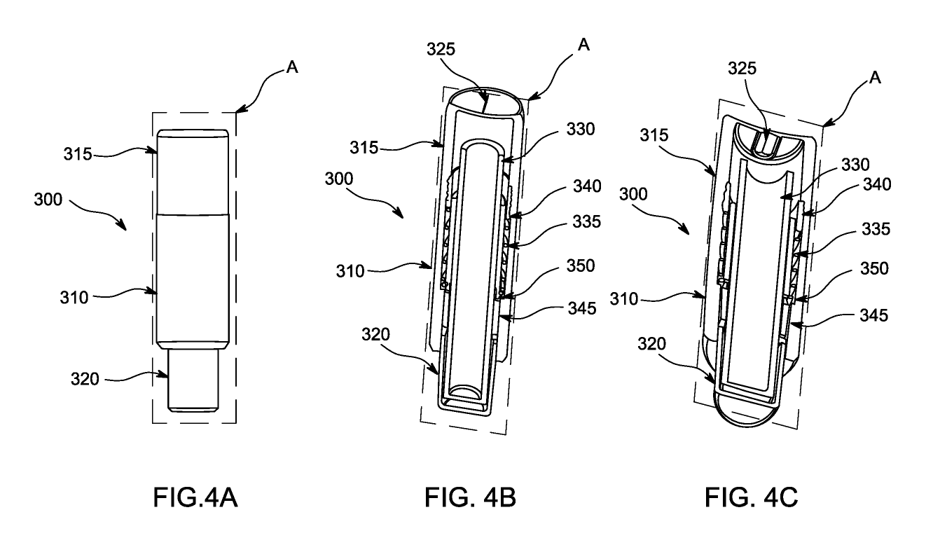

Referring to FIG. 4A the exterior of the rechargable topical fluid applicator 300 is shown having the cap 315, housing 310, and push member 320. The topical fluid applicator 300 is defined by a cross-sectional plane A. And, FIGS. 4B and 4C show the internal components of the topical fluid applicator 300 according to the sectional view of plane A.

FIGS. 4B and 4C show the receptacle 330 for holding a replaceable topical fluid applicator cartridge (not shown). The receptacle 330 is configured for reciprocation within the housing 310 between a protracted position and a retracted position relative to the housing 310. The protracted position of the receptacle 330 being an upward or distal position nearest the tip of the cap 315 where a sealing feature 325 is disposed. The retracted position being a downward or proximal position within the housing 310 further from the tip of the cap 315 where the sealing feature 325 is disposed.

The topical fluid applicator 300 further comprises a spring 335 biasing the receptacle 330 in the retracted position. The spring 335 is held within the housing 310 between an upper bearing member 340 secured to the housing and a lower spring mount of a ratchet mechanism 345.

The topical fluid applicator 300 further includes the push member 320 for moving the reciprocating receptacle 330 into the protracted and retracted positions in cooperation with the ratchet mechanism 345 for holding the receptacle 330 in the protracted position and for releasing the receptacle 330 from the protracted position to the retracted position.

The seal 325 includes a resilient opening having sealing lips extending substantially across a diameter of the width of the lid 315 thereby creating a displaceable opening for the topical fluid applicator to extend there through in the protracted position. Once, retracted the interface between the lips of the seal 325 isolates the applicator from accidental contact, the external atmosphere, and contamination.

Relatively near the a distal end of the receptacle 330, there is formed an annular abutment of the bearing member 340 to cooperate with one end of a helical compression spring 335 which encircles the receptacle 330 and bears at its other end upon an internal opposing annular abutment 350 formed in the rotatable ratcheting mechanism 345. The arrangement is such that when the receptacle 330 is controlled by the push member 320 the rotatable ratcheting mechanism 345 in cooperation with the push member 320 the receptacle 315 is displaced axially from an operative protracted position in which an applicator head 360 (see FIGS. 3B and 3C) of the replaceable cartridge 350 protrudes through the opening 325 in the cap 315 to a sufficient degree to enable it to be employed for the application of topical fluid and to the retracted position in which the application head 360 is retracted within the cap 315 and housing 310. The displacement of the applicator 360 to the operative protracted position is effected against the action of the compression spring 335 and the retraction of the application head 360 is effected under the action of the pressure of the spring 335 with cross reference to FIGS. 3A, 3B, 3C, and 3D.

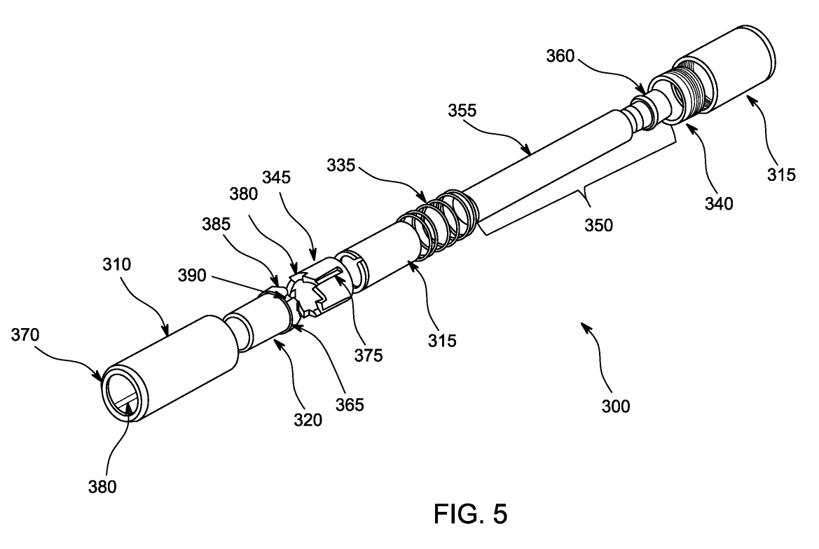

FIG. 5 shows an exploded view of the rechargeable topical fluid applicator 300 along with a replaceable cartridge 350 having a reservoir 355 and application head 360 inserted therein. Thus, FIG. 5 illustrates an embodiment of the topical fluid applicator in combination with the replaceable cartridge 350 as well as methods for manufacture and assembly thereof.

The housing 310 in connection with the cap 315 are collectively of shorter axial length than the replaceable cartridge 350. This is so that the cartridge 350 held by the receptacle 315 and extending into the push element may be readily placed within or extending from the cap and hosing as mechanically manipulated by the push element 320 in combination with the retraction mechanism 345.

In this embodiment, the cap 315 is made of a resilient material such as silicone and includes internal threads cooperating with external threads of the bearing element 340 which is secured to the hosing 310 thereby functionally containing the components therein upon removal of the lid 315. Thus, the cap 315 may be unscrewed from the bearing element to remove the cartridge 350 from the receptacle 315. Once a replacement cartridge 350 is inserted therein the cap 315 is refastened to the bearing element 340 using the cooperating screw threads.

The ratchet element 345 is a rotatable member consisting of a hollow cylinder having a plurality of axially extending slots 375. The push element 320 is closed at one end and formed at the opposing end with an external lip 365 that engages an internal lip 370 of the housing 310 preventing the push member 320 from moving beyond the ends of the housing 310 when the lip 365 of the push member 320 engages the lip 370 of the housing 310. The housing has a plurality internally extending radial projections 380 corresponding to the plurality of axially extending slots 375 of the ratchet element 345 and corresponding with a plurality of axially extending slots 390 of the push element 320. The plurality of projection elements 380 may also be called a lug element or alignment protrusion, and there can be three radial projection elements in the illustrated embodiment.

The body of the ratchet element 345 is adapted to be received slidably within the housing 310 against the helical compression spring 335 which bears at one end on bearing element 340 and at its other end on the ratchet element 345 so that teeth 380 of the ratchet element 345 are normally urged into engagement with teeth 385 of the push element 320.

The ratchet element 345 is closely held within the housing 310. The housing 310 has its internal surface formed at the inner end to a cylindrical shape adapted to receive the cylindrical outer surface of the ratchet element 345 at a relatively close fit. The slots 390 on the push element 320 are of such a length that a short portion of each of their outer ends would normally rest on the inner end face of the interior of the housing 310. This end face of the push element 320 is formed with a plurality, for example six, ratchet-like teeth 390, each having one flank contained in a plane also containing the axis of the member and its other flank inclined relatively steeply. Three longitudinally extending protrusions 380 are formed in the inner surface of the housing 310 equally spaced around the axis thereof so that each protrusions 380 registers with the lower end of the inclined flank of one of the teeth 390. These protrusions 380 are of such a depth that the ratchet member 345 may slide into the housing 310 when its grooves 390 are in line with the protrusions 380 of the housing 310 and protrusions 380 extend from the lower ends of the inclined flanks of alternate teeth 385.

The ratchet member 345, while being slidable in the housing 310, is prevented from rotating relative thereto by forming the ratchet member over its cylindrical length with the one or more longitudinal grooves 375 each adapted to engage in a corresponding protrusion 380 in the annular housing 310. The inner end face of the ratchet member 345 is formed with the teeth 380 which are the same in number and of the same character as those teeth 390 formed on the end of the push member 320, but are so disposed that they are displaced through half a tooth pitch in relation to those teeth around the cam of the two members 320 and 345. As will be understood the grooves 375 on the ratchet member 345 are engaged by the inclined faces of teeth 385 on the push member 320 when this ratchet member 345 is displaced inwardly to a sufficient extent.

The operation of the mechanism of the topical fluid applicator 300 described is as follows. The grooves 375 on the ratchet member 345 are engaged in the corresponding protrusions 380 in the housing 310 when no pressure is applied to the push member 320. The pressure of the spring 335 urges the receptacle 315 and cartridge 350 axially to force the ratchet member 345 further into the housing 310, compressing the spring 335 until the grooves 390, by sliding down the projections 380, i.e. toward the application head 360 of the cartridge 350, eventually come to rest on the inclined faces of the appropriate teeth 385 on the push member 320. In this position the application head 360 is retracted within the cap 315 of the topical fluid applicator 300.

Upon manual pressure exerted against the push member 320 the push member 320 is axially displaced inwards of the housing 310. The ratchet member 345 is moved axially of the housing 310 and the receptacle 315 and the cartridge 350 are correspondingly urged axially forwards against the action of the spring 335 to project the application ball of the application head 360 through the sealing element 325 (see FIGS. 4B and 4C) in the end of the cap 315. When the grooves 375 on the ratchet member 345 have passed completely out of the protrusions 380 of the housing 310, they are supported merely by the inclined flanks of the respective teeth 385 on the push member 320. Under the pressure of the spring 335 the grooves 375 on the ratchet member 345 tend to slide down the inclined flanks on the teeth 385 on the push member 320 into engagement with the respective axially disposed flanks of the corresponding teeth 380 on the ratchet member 345. As the displacement of the push member 320 is continued the lower ends of the inclined flanks of the teeth 385 on the push member 320 come into alignment with the upper ends of the corresponding flanks on the teeth 380 of the reciprocation member 345. The projections of the teeth 380 continue their sliding motion down the inclined flanks, until they meet the axial flanks of the adjacent teeth 385 on the push member 320, the reciprocation member 345 rotating slightly about its axis. Upon releasing the pressure on the push member 320, the pressure of the spring 335 returns the receptacle 315 and cartridge 350 to the retracted position but the reciprocation member 345 is prevented from moving so long as the crests of the teeth 385 on the push member project above the inclined flanks of the teeth 380 on the ratchet member 345.

When it is desired to retract the applicator head 360 of the cartridge 350 once again, pressure is exerted on the push member 320 inwards. As before, and as shown in FIGS. 3A, 3B, 3C, and 3D, the inclined flanks of the appropriate teeth 385 on the push member 320 eventually contact the projections of the teeth 380 on the ratchet member 345 upon which they were resting. When the displacement has reached the stage at which the inclined flanks on the two sets of teeth 385 and 380 come into alignment with each other, the projections slide down the inclined flanks, this sliding motion initially being for half the length of the face concerned owing to the projections coming against the axial flanks on the teeth 385 on the push member 320. When the push member 320 is released, the sliding motion is continued (as soon as the push member 320 has returned through a short distance) and the grooves 390 of the push member 320 arrive at the end of the protrusions 380 formed in the inner wall of the housing 310. At this point the grooves 390 slide down the protrusions 380 so that the receptacle 315 and cartridge 350 can move axially under the action of the spring 335 to retract the applicator 350 point within the cap 315 and housing 310. At this point, the push member 320 reaches its original position.

It will be understood that at each operation of the mechanism, the application head is projected from the lid to a slightly greater extent than is finally required for the application position and also rotates through an angle defined by the mating of the teeth 380 of the ratchet member and the teeth 385 of the push member 320. Moreover, it will be appreciated that repeated reciprocations of the push member 320 will result in intermittent unidirectional rotation of the ratchet member 345 through a series of positions.

In addition to the sealing feature, application had, and other novel features disclosed herein, the novel mechanism described is also an improvement at least in that the cartridge 350 can be removed and replaced without disassembly of the retraction mechanism. That is, the particularly simple and reliable in operation of the retraction mechanism is intact even with removal of the cap 315 and cartridge 350 therefrom. Thus, it is made an additional improvement according to the teachings herein that replacement of the cap 315 can be removed for the purpose of changing a cartridge 350 without complete disengagement of the ratchet element 345 from the push element 320 and spring 335.

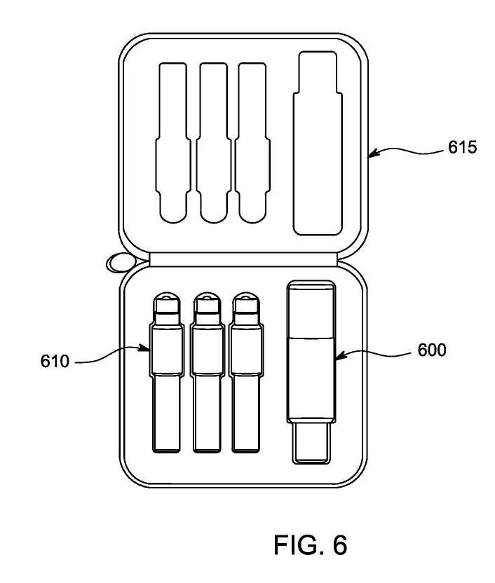

FIG. 6 illustrates a kit having a retractable topical fluid applicator device 600, a plurality of topical fluid applicator cartridges 610 for interchangeable placement in the retractable topical fluid applicator device 600 and a case 615. In this example, the case 615 is a molded clamshell case having molded impression for holding the retractable topical fluid applicator device 600 and the plurality of topical fluid applicator cartridges 610 in place.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It is understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

As will be understood by one skilled in the art, for any and all purposes, such as in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, quadrants, thirds, etc. As a non-limiting example, each range discussed herein can be readily broken down into a lower third, middle third and upper third, etc. As will also be understood by one skilled in the art all language such as “up to,” “at least,” and the like include the number recited and refer to ranges which can be subsequently broken down into subranges as discussed above. Finally, as will be understood by one skilled in the art, a range includes each individual member. Thus, for example, a group having 1-3 routes refers to groups having 1, 2, or 3 routes. Similarly, a group having 1-5 impact zones refers to groups having 1, 2, 3, 4, or 5 impact zones and more or less, and so forth.

From the foregoing, it will be appreciated that various embodiments of the present disclosure have been described herein for purposes of illustration, and that various modifications may be made without departing from the scope and spirit of the present disclosure. Accordingly, the various embodiments disclosed herein are not intended to be limiting, with the true scope and spirit being indicated by the following claims. All references recited herein are incorporated herein by specific reference in their entirety.

What is claimed is:

1. A retractable topical fluid applicator device, comprising:
a housing;
a receptacle for holding a topical fluid applicator cartridge, the receptacle being configured for reciprocation within the housing between a protracted position and a retracted position;
a spring biasing the receptacle in the retracted position;
a push member for moving the reciprocating receptacle into the protracted position;
a mechanism for holding the receptacle in the protracted position and for releasing the receptacle from the protracted position; and
a seal disposed at an end of the housing.

2. A retractable topical fluid applicator device according to claim 1, further comprising the topical fluid applicator cartridge held by the receptacle, wherein the topical fluid applicator extends through the seal in the protracted position and is covered by the seal in the retracted position.

3. A retractable topical fluid applicator device according to claim 2, wherein the topical fluid applicator cartridge is held by a slip engagement with the receptacle for replacement with a different topical fluid applicator cartridge.

4. A retractable topical fluid applicator device according to claim 2, the topical fluid applicator cartridge including a topical fluid applicator head, a reservoir, and a viscous oil contained within the reservoir.

5. A retractable topical fluid applicator device according to claim 4, wherein the topical fluid applicator head comprises:
a barrel including:
an interior surface and an outer surface;
a support appendage extending around a periphery of the cylindrical interior surface;
an applicator ball disposed within the barrel and supported by the annular support appendage of the barrel.

6. A retractable topical fluid applicator device according to claim 5, wherein the topical fluid applicator head further comprises:
a sealing ring disposed around an upper periphery of the ball;
a can including:
an inner surface corresponding to the outer surface of the barrel; and
an annular lip extending over a top surface of the sealing ring thereby holding the sealing ring against the applicator ball and exposing a portion of the applicator ball for application of the topical fluid; and
a biasing member, the biasing member urging the applicator ball against the sealing ring.

7. A retractable topical fluid applicator device according to claim 1, wherein a height of the topical fluid applicator device is at least 60 millimeters and a width of the topical fluid applicator device is at least 15 millimeters.

8. A retractable topical fluid applicator device according to claim 1, wherein the seal includes a resilient slit having sealing lips extending substantially across a diameter of the width of the seal thereby creating a displacable opening for the topical fluid applicator to extend through in the protracted position and a sealed interface between the lips when the topical fluid applicator is in the retracted position.

9. A retractable topical fluid applicator device according to claim 1, wherein:
the push member is non-rotatable and includes downwardly projecting teeth;
the mechanism for holding the receptacle in the protracted position and for releasing the receptacle from the protracted position including a rotatable ratchet interlocking member.

10. A retractable topical fluid applicator device according to claim 9, the ratchet member having upwardly projecting teeth which selectively engage the downwardly projecting teeth of the push member.

11. A retractable topical fluid applicator device according to claim 9, the ratchet member being disposed between the spring and the push member whereby the spring forces the rotatable ratchet member against the teeth of the push member, at least one of the engaging surface of the teeth of the push member and the rotatable ratchet member being inclined so that the force of engagement between the teeth of the push member and the engaging surface of the rotatable ratchet member caused by the spring rotatably biases the rotatable member ratchet member for selective transition between the protracted and retracted positions.

12. A topical fluid applicator head, comprising:
a barrel including:
an interior surface and an outer surface;
a support appendage extending around a periphery of the cylindrical interior surface;
an applicator ball disposed within the barrel and supported by the annular support appendage of the barrel;
a sealing ring disposed around an upper periphery of the ball;
a can including:
an inner surface corresponding to the outer surface of the barrel; and
an annular lip extending over a top surface of the sealing ring thereby holding the sealing ring against the applicator ball and exposing a portion of the applicator ball for application of the topical fluid; and
a biasing member, the biasing member urging the applicator ball against the sealing ring.

13. A topical fluid applicator head according to claim 12, further comprising a holder disposed between the biasing member and the applicator ball, the biasing member pressing the holder against a lower surface of the applicator ball.

14. A topical fluid applicator head according to claim 12, the sealing ring creating an annular seal around an upper periphery of the applicator ball.

15. A topical fluid applicator head according to claim 12, the biasing member comprising a wave washer.

16. A topical fluid applicator head according to claim 12, further comprising a cap, the cap disposed over the can and applicator ball.

17. A topical fluid applicator head according to claim 16, wherein the cap extends over the applicator ball without contacting the applicator ball.

18. A topical fluid applicator head according to claim 12, further comprising an insertion sleeve extending below the annular support for interference mounting the barrel to a reservoir.

19. A topical fluid applicator comprising the topical fluid applicator head according to claim 12, further comprising a reservoir of fluid interference fit to the insertion sleeve, the reservoir providing the topical fluid to the applicator ball for rotational application of the fluid to the skin of a human.

20. A kit comprising:
a retractable topical fluid applicator device, including:
a housing;
a receptacle for holding a topical fluid applicator cartridge, the receptacle being configured for reciprocation within the housing between a protracted position and a retracted position;
a spring biasing the receptacle in the retracted position;
a push member for moving the reciprocating receptacle into the protracted position;
a mechanism for holding the receptacle in the protracted position and for releasing the receptacle from the protracted position; and
a seal disposed at an end of the housing;
a plurality of the topical fluid applicator cartridges for interchangeable placement in the receptacle of the retractable topical fluid applicator device.
